(19) United States
(12) Patent Application Publication
(10) Pub. No.: US 2001/0049103 A1
(43) Pub. Date: Dec. 6, 2001

(54) Platform for the discovery of the bacterial genes involved in RNA modification

(76) Inventors: T. Guy Roberts, Oakland, CA (US); Wayne Mitchell, San Francisco, CA (US); Kenneth Beckman, Alameda, CA (US)

Correspondence Address: LAW OFFICES OF JONATHAN ALAN QUINE, P O BOX 458, ALAMEDA, CA, 94501

(21) Appl. No.: 09/792,437

(22) Filed: Feb. 23, 2001

Related U.S. Application Data
(60) Provisional application No. 60/185,071, filed on Feb. 25, 2000.
(60) Provisional application No. 60/185,000, filed on Feb. 25, 2000.
(60) Provisional application No. 60/225,505, filed on Aug. 15, 2000.
(60) Provisional application No. 60/225,506, filed on Aug. 15, 2000.

(51) Int. Cl. C12Q 1/68; C12Q 1/00; C07H 21/04

(52) U.S. Cl. 435/6; 435/4; 536/23.7

(57) ABSTRACT

Methods for identifying genes and gene products involved in RNA modification, and methods for screening test compounds or antibiotics for activity are provided. Additionally, the gene products identified in the methods, the genes that encode the gene products, modified sentinel molecules produced by the gene products, and compounds or antibiotics which inhibit the modification process are provided.

### CROSS-REFERENCE TO RELATED APPLICATIONS

This application is related to U.S. Ser. No. 60/185,071, filed Feb. 25, 2000, U.S. Ser. No. 60/185,000, also filed Feb. 25, 2000; U.S. Ser. No. 60/225,505, filed Aug. 15, 2000; and U.S. Ser. No. 60/225,506, also filed Aug. 15, 2000. The present application claims priority to, and benefit of, these applications pursuant to 35 U.S.C. § 119(e).

### BACKGROUND OF THE INVENTION

The development of antibiotic resistance is a natural process in bacteria, commonly achieved through the acquisition of mechanisms to expel the antibiotic from the cellular system, or to modify it into a less toxic form. However, the prevalent and often casual use of currently-available antibiotics has accelerated this process, leading to multiply-resistant bacterial strains. This rapid development of antibiotic resistance can be delayed by a more selective approach to the application of antibiotics, but is unlikely to be avoided entirely. Therefore, the identification and development of new antibiotics will continue to be necessary. The present invention meets these and other needs by providing new antibiotic targets, and antibiotic and antibiotic target discovery platforms.

### SUMMARY OF THE INVENTION

Discovery of therapeutic agents is facilitated by an understanding of the function of genes whose gene products represent safe and effective drug targets. Assigning an enzymatic activity to a gene product, protein or RNA, can be difficult without a lengthy investigation into the substrates of the catalyzed reaction. Although it can be possible to assign a gene's product to a particular class of enzymes by computational and comparative means, the specific activity often remains unknown without biochemical or genetic studies. Moreover, screening procedures that seek to identify an organism's essential genes can not reveal the function of these genes. The methods of the present invention differ from other drug target screening procedures in that these methods can be used to identify the genes directly responsible for a particular biosynthetic activity, for example, RNA modification activity. The expression of a candidate ‘test’ gene is modulated within an organism and the product of the activity of interest is analyzed for similar modulation. Assays based on detection of the products of a reaction, whether enzymatic modification of a biomolecule or general detection of substrate-to-product conversion, necessarily links the gene of interest (e.g., the test gene) to a particular function or activity. Furthermore, if this activity is vital to the pathogenicity of an organism or a disease, then the identification of the responsible enzyme and its gene in the above manner serves to characterize a useful drug target. The present invention systemizes this process of drug target identification by providing methods for correlating molecular and cellular structures to their causative genes. Furthermore, this invention provides methods for the simultaneous discovery of classes of enzymes that share at least one substrate in common. Where the members of the chosen class of substrates, herein termed “sentinel molecules,” can be modified by any of a number of catalytic mechanisms, the assay is not limited to a specific enzymatic activity. When performed in a multi-well format, the methods employing these sentinel molecules can be performed in a high throughput fashion. Thus, it is an object of the invention to provide a platform of methods upon which modifications of a sentinel molecule, such as one or more transfer RNA (tRNA) molecules, can be examined and optionally correlated to the presence, absence, or expression of a particular gene. This platform can be used, for example, to identify whether the sentinel molecule is modified, the type of modification present, the genes involved in the modification, the gene products which execute the modification, and one or more test compounds which affect the occurrence and/or extent of the modification. Within the assay solution, the sentinel molecule can be modified by the gene product in a number of ways, including, but not limited to, methylation, alkylation, acetylation, esterification, ubiquitination, lysinylation, phosphorylation, sulfation, glycosylation, or a combination thereof. The presence and extent of these modifications can be determined by one or more of a variety of analytical techniques, such as mass spectrometry, thin layer chromatography, HPLC, capillary electrophoresis, NMR spectroscopy, X-ray crystallography, infrared spectroscopy, or cryo-electron microscopic analysis.

Accordingly, the present invention also provides methods for screening a test compound for activity, comprising the steps of preparing an assay solution having a gene product (for example, an enzyme or a catalytic RNA) capable of modifying a sentinel molecule; incubating the assay solution with the sentinel molecule and a test compound; and determining whether the sentinel molecule was modified by the gene product in the presence of the test compound, thereby screening the test compound for activity. The test compound can be, for example, an antibiotic compound. The sentinel molecule can be any of a number of cellular components, including, but not limited to, various RNA molecules (for example, tRNA, rRNA, mRNA, guide RNA, snRNA molecules, snoRNA molecules, and hnRNA molecules), DNA molecules, peptides, proteins, carbohydrates, lipids, naturally-occurring small molecule substrates, and synthetic small molecule substrates. The assay solution is optionally a cellular extract, derived from either bacterial sources or eukaryotic sources. Alternatively, the assay solution is a solution containing a purified enzyme and one or more substrates (in, for example, purified or synthetic forms, or in crude mixtures, i.e., processed cellular lysates).

The present invention also provides methods for screening a compound for antibiotic activity. The antibiotic screening method includes the steps of providing a cell line comprising a sentinel RNA molecule that is normally modified in a prokaryotic system, but not modified in a eukaryotic system; treating the cell line with the compound to be screened; and monitoring the sentinel RNA molecule for modification. Depending on the design of the assay, either a single compound or multiple compounds can be screened for antibiotic activity using the methods provided. The gene to be tested can be expressed in a cell line or tissue of interest, or the gene can be genetically engineered into tissues or cell lines of eukaryotic, archae or eubacterial origin. The gene product can be assayed in vitro or in vivo. The sentinel molecule can be any type of molecule, but is described here as a tRNA of natural or synthetic source. The assay solution can be either a cellular extract or a solution of defined components, within which the sentinel RNA molecule can be modified in a number of ways, including, but not limited to, methylation, alkylation, acetylation, esterification, ubiquitination, lysinylation, phosphorylation, sulfation, glycosylation, or a combination thereof. The presence and extent of these modifications can be determined by one or more of a variety of analytical techniques, such as mass spectrometry, thin layer chromatography, HPLC, capillary electrophoresis, NMR spectroscopy, X-ray crystallography, or cryo-electron microscopic analysis.

In addition, the present invention provides in vitro methods for identifying one or more gene products involved in RNA modification. The in vitro methods include the steps of providing at least one cell having one or more test genes that encode at least one gene product; preparing a cellular extract from the cell, such that the cellular extract contains the gene product; incubating the cellular extract with a sentinel RNA molecule; and determining whether the sentinel RNA molecule has been modified by the gene product, thereby determining whether the gene product (and by association, the test gene) participates in an RNA modification process. The cellular extract can be derived from, for example, a bacterial cell or a eukaryotic cell. The test gene can be a part of the cellular genome, or it can be introduced into the cell by a number of techniques, for example, via an expression vector. The expression level of the gene product can be altered for use in the method of the present invention, particularly if generation of a particular genotype, or particular phenotype, is desired. The expression level can, for example, be induced, increased, reduced, or even eliminated. The sentinel RNA molecule can be any of a number of RNA molecules, including, but not limited to, tRNA, rRNA, mRNA, guide RNA, snRNA molecules, snoRNA molecules and hnRNA molecules. The assay solution is optionally a cellular extract, within which the sentinel molecule can be modified in a number of ways, including, but not limited to, methylation, alkylation, acetylation, esterification, ubiquitination, lysinylation, phosphorylation, sulfation, glycosylation, or a combination thereof. The presence and extent of these modifications can be determined by one or more of a variety of analytical techniques, such as mass spectrometry, thin layer chromatography, HPLC, capillary electrophoresis, NMR spectroscopy, X-ray crystallography, or cryo-electron microscopic analysis. The methods can further include the step of identifying the test gene or genes that encode the gene product that performed the modification.

Furthermore, the present invention provides in vivo methods for identifying one or more gene products involved in RNA modification. The in vivo methods include the steps of providing a cell having at least one sentinel RNA molecule, and one or more test genes of interest; manipulating the test gene (e.g., altering the expression of the gene product); and monitoring the sentinel RNA molecule for modification by the one or more gene products encoded by the one or more test genes, thereby determining whether the gene product (and by association, the test gene) participates in an RNA modification process. By manipulating the test gene present in the in vivo tissue, expression of the gene product can be induced, increased, reduced, or eliminated. The in vivo method can further include monitoring whether the gene product encoded by the test gene is increased, reduced or eliminated. The sentinel RNA molecule can be any of a number of RNA molecules, such as tRNA, rRNA, mRNA, guide RNA, snRNA molecules, snoRNA molecules, and hnRNA molecules. The presence and extent of the modifications to the sentinel RNA molecule can be determined by one or more of a variety of analytical techniques, such as HPLC, mass spectrometry, liquid chromatography/mass spectrometry (LC/MS), thin layer chromatography, capillary electrophoresis, NMR spectroscopy, X-ray crystallography, or cryo-electron microscopic analysis. The methods can further include the step of identifying the test gene or genes that encode the gene product.

The present invention also provides methods of identifying a gene encoding a desired gene product. The methods include providing a library of nucleic acids and expressing the library to provide a plurality of gene products for analysis. The plurality of gene products are incubated with one or more sentinel molecules and the resulting products are analyzed for the presence or absence of one or more modifications to one or more of the sentinel molecules. Using the methods of the present invention, it can be determined whether the plurality of gene products includes one or more desired gene products, and the gene encoding the desired gene product is identified. The desired gene product can be any of a number of proteins, enzymes, RNA molecules, and the like.

The present invention also provides the modified sentinel molecules generated during the methods of the present invention, the gene products which perform these modifications, the genes that encode these gene products, and the test compounds and/or antibiotics which, for example, enhance or inhibit the modification of the sentinel molecules.

### BRIEF DESCRIPTION OF THE DRAWING

FIG. 1 illustrates various methodologies for the analysis of gene function.

FIG. 2 is a flowchart depicting one embodiment of the methods of the present invention.

FIG. 3 depicts an in vitro assay as performed by the methods of the present invention.

### DETAILED DISCUSSION OF THE INVENTION

Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, reference to “a sentinel molecule” includes mixtures of sentinel molecules, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

As used herein, the term “sentinel molecule” refers to one or more molecules that can be monitored for change in structure (e.g., by the addition or removal of one or more atoms) or change in one or more physical properties (e.g., the ability to bind an enzyme), usually within the context of a cellular system. The sentinel molecule can be a molecule that naturally occurs in the biological system being examined, or it can be a molecule that is added to the system for the purpose of monitoring, for example, a specific enzymatic activity.

The term “test compound” refers to a compound which is being added to an assay system to assess the effect that the compound has upon the assay system. The test compound can be a synthetic compound (i.e. prepared by chemical synthesis or chemical modification), or it can be a naturally-occurring compound. As used herein, a test compound is meant to encompass both a single compound, as well as a group, or “library,” of compounds.

The term “test gene” refers to one or more nucleic acid sequences that encode one or more gene products. The test gene can be a portion of a cellular genome, an isolated DNA sequence, or a synthetically prepared or artificially manipulated sequence. The test gene can be part of the cellular genome, or it can be external to the cellular genome (for example, a component of an expression vector). In addition, the test gene can be one or more components of a library of sequences.

The term “gene product” refers to product encoded by one or more genes. The gene product can be a protein, an RNA molecule, or a DNA molecule.

Discovery of Therapeutic Targets and Novel Drug Compounds

Pharmaceutical companies are pursuing new antimicrobial drug targets by several methods, including the random screening of microbial genes for lethality upon disruption, for gene expression, or for activity upon exposure to compound libraries. An alternative approach to the identification of cellular targets having therapeutic value (for example, targets that demonstrate a sensitivity to antibiotics) is to place the focus of the screens on the products of the reactions catalyzed by the gene products and assay for the effect of modulation of “test genes” on the presence or abundance of the covalent modification on the “sentinel molecule.” Often, these modifications (and the gene products that generate them) will differ among viral, prokaryotic and eukaryotic systems, or among members of these groups (for example, among different types of prokaryotes), providing some selectivity of the therapeutic target.

The present invention provides methods for screening test compounds, as well as methods for screening compounds for antibiotic activity. In addition, the present invention provides both in vitro as well as in vivo methods for identifying gene products involved in RNA modification. A common component to these methods is the use of a “sentinel molecule” for the purpose of, for example, monitoring the activity of one or more gene products. In the in vitro and in vivo methods, the sentinel molecule is used to identify gene products of interest (i.e., therapeutic targets), by determining whether the sentinel molecule has been modified in the presence of the gene product. Alternatively, in the screening methods, the assay systems are examined for a change (for example, an enhancement or inhibition) in the modification of the sentinel molecule, thus indicating whether a test compound or a putative antibiotic has an effect on the cellular metabolism. These assays can be performed in a high-throughput manner, such that libraries of compounds, or collections of gene sequences, can be tested for involvement in microbial metabolism, and optionally RNA modification. These methods, and the sentinel molecules employed in these methods, are described in further detail below.

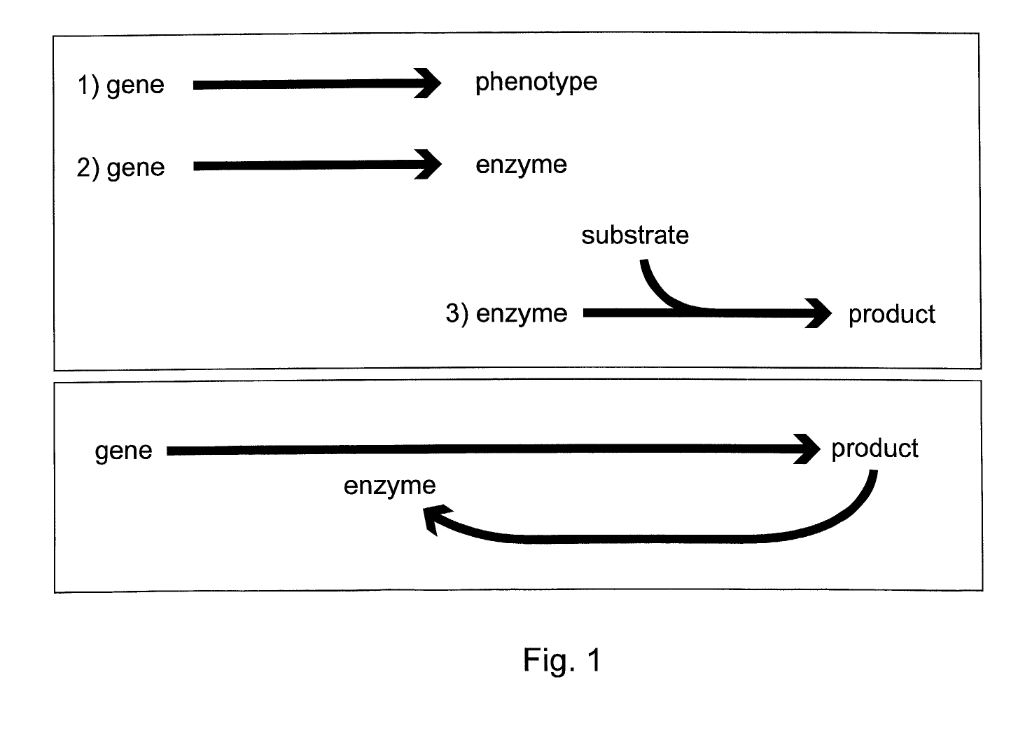

FIG. 1 depicts various methodologies for the analysis of gene function, according to one class of embodiments of the present invention. For example, as depicted in the upper panel, gene function is commonly determined by looking at a phenotype of the cell or organism. Once the phenotype is known, the gene is then isolated, and the resulting gene product is shown to be capable of converting a defined substrate into an expected product. In the methods of the present invention, gene function is typically determined without the intervening step of analysis of the gene product and without the need for phenotype information. The gene is simply expressed (e.g., in an expression library format) and the cells or cellular components are directly assayed for the ability of the expressed gene product to convert a known substrate (such as an unmodified RNA) to a particular product (e.g., a modified tRNA molecule). Expression of the gene can occur in vitro (e.g., using a transcription-translation faormat) or in vivo (e.g., in cells of a library of interest).

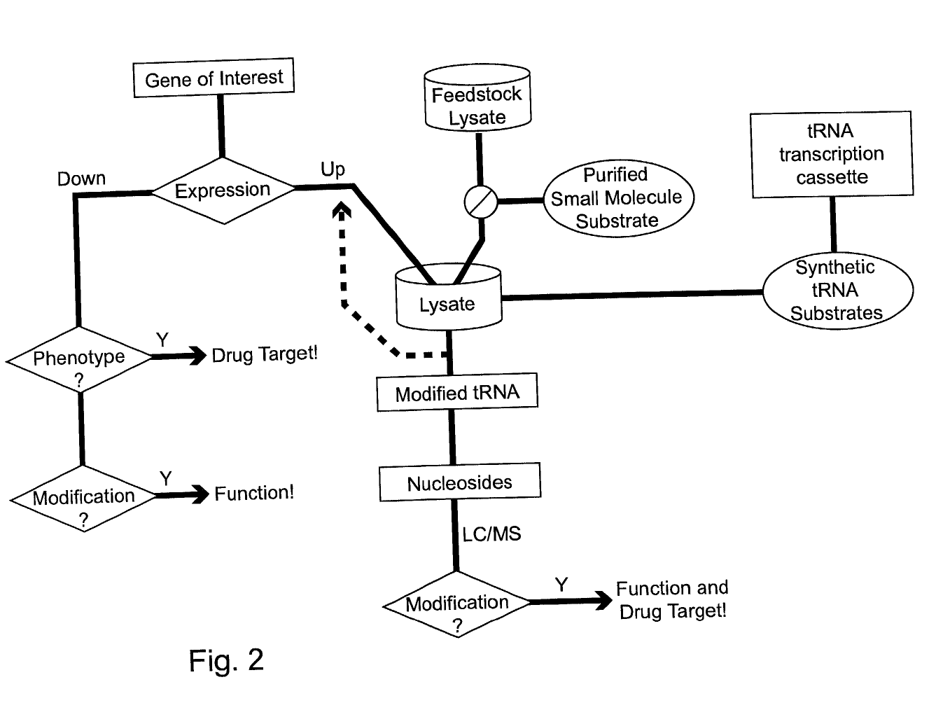

FIG. 2 depicts one embodiment of the methods for identifying a gene encoding an RNA modification enzyme as provided by the present invention. A gene of interest (e.g., a member of a nucleic acid library) is expressed in either a cell-based or in vitro system, and the expression system is then modulated. If the expression is down-regulated, and the phenotype or the desired modification of the sentinal molecule is altered (e.g., if the RNA modification does or does not occur, and the cell gains or loses enzymatic functionality), then the function of the gene as an RNA modification enzyme is identified and the gene or gene product is potentially of interest as a drug target. Optionally, the gene expression is up-regulated and the cellular lysate is combined with synthetic tRNA substrates (i.e. the unmodified sentinal molecule), and optionally one or more small molecule substrates. The resulting products (the modified tRNA molecules) are digested to component nucleosides (or nucleotides), which are then analyzed by LC/MS for the presence or absence of one or more modifications. The presence of an RNA modification indicates that the gene being over-expressed functions as an RNA modification enzyme, and is a potential drug target.

Sentinel Molecules

A sentinel molecule is any molecule that can be monitored for change in structure (e.g., by the addition or removal of one or more atoms) or change in one or more physical properties (e.g., the ability to bind an enzyme). The sentinel molecule can be a molecule that naturally occurs in the biological system being examined, or it can be a molecule that is added to the system for the purpose of monitoring, for example, an enzymatic activity. Assays employing sentinel molecules can use a single type of sentinel molecule, a set of structurally similar sentinel molecules, or a group of structurally diverse sentinel molecules; the term “sentinel molecule” is intended to cover all of these possibilities.

Examples of general classes of sentinel molecules include, but are not limited to, RNA molecules, DNA molecules, peptides, proteins, carbohydrates, lipids, naturally-occurring small molecule substrates, and synthetic small molecule substrates. One preferred class of sentinel molecule employed in the methods of the present invention includes sentinel RNA molecules. Examples of RNA molecules that can act as sentinel molecules include, but are not limited to, transfer RNA (tRNA) molecules, ribosomal RNA (rRNA) molecules, messenger RNA (mRNA) molecules, guide RNA molecules, heterogeneous nuclear RNA (hnRNA) molecules, small nuclear RNA (snRNA) molecules, small nucleolar RNA (snoRNA) molecules, and the like. Another class of sentinal molecules employed in the methods of the present invention includes lipid-based molecules, including, but not limited to, glycolipids, phospholipids, triglycerides, lipopolysaccharides, lipoproteins, mycolic acids, teichoic acids, teichuronic acids, lipoteichoic acids, and the like. Carbohydrate-containing sentinal molecules can also be employed in the methods of the present invention; carbohydrate-based sentinal molecules include, but are not limited to, carbohydrates, glycoproteins, glycolipids, lipopolysaccharides, peptidoglycans, fucoidans, and the like.

Cells and Cell Lines

The cells used in the methods of the present invention include either bacterial cells as well as eukaryotic cells. Examples of bacterial cell lines which can be used in the methods of the present invention include, but are not limited to, those from the genuses Aquifex, Archaeoglobus, Bacillus, Borrelia, Chlamydia, Escherichia, Helicobacter, Heliobacterium, Haemophillus, Methanobacterium, Methanococcus, Mycobacterium, Mycoplasma, Pyrococcus, Rickettsia, Synechocystis, and Treponema (See, for example, the lists of microorganism genera provided by DSMZ-Deutsche Sammlung von Mikroorganismen und Zellkulturen GmbH, Braunschweig, Germany, at http://www.dsmz.de/species). Alternatively, eukaryotic cell lines, including mammalian (for example, murine, rodent, guinea pig, rabbit, canine, feline, primate or human cells), amphibian, reptile, fish, nematode, fungal, and plant cells, can be employed.

In addition to clinical and/or environmental samples, cells for use in the methods of the present invention are available from cell repositories such as the American Type Culture Collection (www.atcc.org), the World Data Center on Microorganisms (http://wdcm.nig.ac.jp), European Collection of Animal Cell Culture (www.ecacc.org) and the Japanese Cancer Research Resources Bank (http://cellbank.nihs.go.jp), and companies such as Clonetics Corporation (www.clonetics.com).

Generally, one of skill in the art is fully able to culture and transfect cells from animals, plants, fungi, bacteria and other cells using available techniques. A variety of cell culture media are described in The Handbook of Microbiological Media, Atlas and Parks (eds.) (1993, CRC Press, Boca Raton, Fla.). References describing the techniques involved in bacterial and animal cell culture include Sambrook et al., Molecular Cloning—A Laboratory Manual (2nd Ed.), Vol. 1-3 (1989, Cold Spring Harbor Laboratory, Cold Spring Harbor, N.Y.); Current Protocols in Molecular Biology, F. M. Ausubel et al., eds., Current Protocols, (a joint venture between Greene Publishing Associates, Inc. and John Wiley & Sons, Inc., supplemented through 2000); Freshney, Culture of Animal Cells, a Manual of Basic Technique, third edition (1994, Wiley-Liss, New York) and the references cited therein; Humason, Animal Tissue Techniques, fourth edition (1979, W. H. Freeman and Company, New York); and Ricciardelli, et al., In Vitro Cell Dev. Biol. (1989) vol.25, pp.1016-1024. Information regarding plant cell culture can be found in Plant Cell and Tissue Culture in Liquid Systems, by Payne et al. (1992, John Wiley & Sons, Inc. New York, N.Y.); Plant Cell, Tissue and Organ Culture: Fundamental Methods by Gamborg and Phillips, eds. (1995, Springer Lab Manual, Springer-Verlag, Berlin), and is also available in commercial literature such as the Life Science Research Cell Culture Catalogue (1998) from Sigma-Aldrich, Inc (St Louis, Mo.) (Sigma-LSRCCC) and the Plant Culture Catalogue and supplement (1997) also from Sigma-Aldrich, Inc (St Louis, Mo.) (Sigma-PCCS).

Genes, Gene Expression, and Gene Products

Genes that encode gene products employed in methods of the present invention can be part of the cellular genome, or they can be added to the cells, for example, in the form of expression vectors. As such, the identity of the genes and the functions of their respective gene products may or may not be defined. The genes can be derived from a library of genomic fragments, such as those publicly or commercially available from a number of sources, for example, Gorilla Genomics (Alameda, Calif.) or Incyte Genomics (Palo Alto, Calif.). Alternatively, nucleic acid sequences for use as genes can be amplified in vitro, synthesized de novo and/or assembled using techniques known to those in the art, such as polymerase mediated, ligation-mediated and combination ligation/ polymerase mediated assembly methods. Custom-synthesized nucleic acid sequences can be ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and the like.

A variety of expression vectors can be employed to deliver the genes of interest and control their expression in bacterial and/or eukaryotic cells, including, but not limited to, viruses, plasmids, episomes, transposons, phages, artificial chromosomes (such as a bacterial or yeast artificial chromosome), and the like. Preferably, the construct further comprises regulatory sequences, including, for example, a promoter, operably linked to the test gene. Exemplary promoters for use in the methods of the present invention include, but are not limited to, the E. coli lac or tip promoter, SV40 promoter, phage lambda PL promoter, and CMV promoter. Large numbers of suitable vectors and promoters are known to those of skill in the art, and are commercially available.

Methods of introducing genetic material into cells, including plant and animal cells, e.g., for cloning and sequencing and/or for expression and selection of encoded molecules are generally available, as are methods of expressing proteins encoded by such nucleic acids (see, for example, Ausubel, supra). Transformation methodologies include, but are not limited to, bacterial-mediated transformation, phage transduction, conjugation, transfection, liposome-mediated transformation, protoplast fusion techniques, particle bombardment, electroporation, and the like.

Several of the methods of the present invention involve alteration of the test gene expression levels. This can be accomplished by a number of mechanisms known to those in the art, such as commercially available expression cassettes, expression vectors, and other transcription regulatory systems. The resulting level of gene product expressed in the cells is increased, reduced, or eliminated, depending upon the treatment performed; upon lysing the cells, the resulting assay solution contains an altered amount of gene product as compared to untreated cells.

References describing nucleic acid manipulation techniques are known in the art, and include, for example, Berger and Kimmel, Guide to Molecular Cloning Techniques, Methods in Enzymology volume 152 (Academic Press, Inc., San Diego, Calif.); PCR Protocols A Guide to Methods and Applications (Innis et al. eds.) (1990, Academic Press Inc., San Diego, Calif.); De Lorenzo and Timis Methods in Enzymology (1994) vol. 235, pp.385-404; Kleckner et al. Methods in Enzymology (1991) vol. 204, chapter 7; as well as Sambrook and Ausubel, both supra. These and other references cited herein describe cell culture techniques and recombinant nucleic acid methodologies appropriate for use in the methods of the present invention.

Modifications to Sentinel Molecules

In the methods of the present invention, the sentinel molecule is modified by one or more gene products, leading to a modified sentinel molecule. The modifications orchestrated by the gene products can cover a range of potential changes to the structure of the sentinel molecule. Modifications to the sentinel molecule can include, but are not limited to, methylation, alkylation, acetylation, esterification, ubiquitination (ubiquitinulation), lysinylation, phosphorylation, sulfation, sulfonation, glycosylation, farnsylation, and the like. Alternatively, modifications to sentinal molecules can be the result of the activities of various transferases, synthases, isomerases, dehydrogenases, and the like.

A preferred class of sentinel molecule employed in the methods of the present invention includes sentinel RNA molecules. Sentinel RNA molecules encompass, but are not limited to, tRNA molecules, rRNA molecules, mRNA molecules, guide RNA molecules, snRNA molecules, snoRNA molecules, and hnRNA molecules. One or more component nucleotides of the sentinel RNA molecules can be modified to generate a modified sentinel molecule.

Known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington DC). Modified RNA components include the following: 2′-O-methylcytidine; N⁴-methylcytidine; N⁴-2′-O-dimethylcytidine; N⁴acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyluridine; 5-carboxymethylaminomethyl-2-thiouridine; dihydrouridine; dihydroribosylthymine; 2′-methyladenosine; 2-methyladenosine; N⁶N-methyladenosine; N⁶, N⁶-dimethyladenosine; N⁶,2′-O-trimethyladenosine; 2-methylthio-N⁶N-isopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N²,N²-dimethyl guanosine; N², 2′-O-dimethyl guanosine; N², N², 2′-O-trimethyl guanosine; 2′-O-ribosyl guanosine (phosphate); 7-methyl guanosine; N2;7-dimethyl guanosine; N²; N^{2;7}-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine. The methods of the present invention or others in the art can be used to identify additional modified RNA molecules.

Another preferred sentinal molecule used in the methods of the present invention is a carbohydrate-based or lipid based molecule. Exemplary sentinal molecules include, but are not limited to, phospholipids, triglycerides, lipopolysaccharides, glycolipids, glycoproteins, peptidoglycans, lipoproteins, mycolic acids, teichoic acids, teichuronic acids, lipoteichoic acids, and the like. As with the RNA-based sentinal molecules described in the previous paragraph, a number of modifications can be made to the carbohydrate-based or lipid based sentinel molecule, such as phosphorylation or sulfation; methylation, alkylation, or acetylation; esterification; and addition of relatively large moieties such as amino acids (e.g., lysine), various carbohydrate structures, and proteins such as ubiquitin. Such modifications to sentinal molecules can be the result of the activities of various transferases, synthases, isomerases, dehydrogenases, and the like.

Analysis of Modified Sentinel Molecules

Methods and techniques for analyzing reaction components and products are varied and well known in the art. Some preferred analytical techniques for use in determining whether the sentinel molecule has been modified, the extent of modification, and/or the type of modification include, but are not limited to, mass spectrometry, thin layer chromatography (TLC), high pressure liquid chromatography (HPLC), capillary electrophoresis (CE), NMR spectroscopy, X-ray crystallography, cryo-electron microscopic analysis, or a combination thereof.

Traditionally, analysis of modifications of sentinel molecules such as RNA molecules has been performed using thin layer chromatographic techniques on radioactive substrates. References exist which address this traditional analytical methodology. More recently, mass spectrometry (MS) has been used by several academic groups to assess the modification states of RNA molecules.

Mass spectrometry is a particularly versatile analytical tool, and includes techniques and/or instrumentation such as electron ionization, fast atom/ion bombardment, MALDI (matrix-assisted laser desorption/ionization), electrospray ionization, tandem mass spectrometry, and the like. A brief review of mass spectrometry techniques commonly used in biotechnology can be found, for example, in Mass Spectrometry for Biotechnology by G. Siuzdak (1996, Academic Press, San Diego).

In the methods of the present invention, the assay solutions (containing the newly modified sentinel molecules) are prepared for mass spectrometry and then transferring the resulting yield of sentinel molecules into a suitable solvent system. Analysis by mass spectrometry yields a spectrogram from which both the mass and composition of the sentinel molecule can be determined, in both the modified or unmodified states. By direct comparison of these spectrograms, the modification state of the sentinel molecule is revealed in a computationally straightforward manner. The presence or absence of modifications on these sentinel molecules determines the relevance of the manipulated test gene to the enzymatic pathway that produces the modifications. Direct analysis of the sentinel molecules is also possible, though this requires a complete analysis of the composition of the unmodified sentinel molecule.

Alternatively, the assay solutions containing the newly modified sentinel molecules are prepared for NMR spectroscopy by removal of the original solvent solution (for example, by lyophilization), and re-dissolution into a stable-isotope solvent, such as a deuterated solvent solution. Suitable deuterated solvent solutions include, but are not limited to D₂O (deuterium oxide), CDCl₃, DMSO-d6, acetone-d6, and the like (available from Cambridge Isotope Labs, Andover, Mass.; www.isotope.com). Optionally, the samples can be analyzed using LC-NMR spectroscopy. Analysis by these methodologies can provide information related to both the presence of one or more modifications, as well as the type or identity of the modification (see, for example, NMR of Macromolecules: A Practical Approach, G. C. K. Roberts, ed., 1993, Oxford University Press, New York).

Screening Test Compounds using Cellular Assay Solutions

One embodiment of the methods of the present invention provides methods for screening a test compound for activity. In these methods, an assay solution is prepared, containing a gene product that is capable of modifying a sentinel molecule. The assay solution can be a cellular extract, prepared from a single cell or a plurality of cells (i.e. a cell line or an in vivo tissue). The gene product can be a protein (for example, an enzyme), a ribonucleic acid sequence (such as a ribozyme), or a deoxyribonucleic acid (for example, a cDNA prepared by reverse transcription, or a PCR product). The gene that encodes the gene product used in the methods can be a gene present in the cellular genome, or it can be a gene present in a structure external to the cellular genome, such as a virus, a plasmid, an expression vector and the like.

In preparing the assay solution, the cells can be treated such that the expression level of the gene product is altered. Manipulation of the expression of the gene product can be performed at the level of the gene or at the level of the gene product. For example, the expression of gene product can be controlled at the gene level through stimulation or inhibition of various transcription activities, alteration of promoters, generation of temperature-sensitive mutations and the like. Genes can be manipulated through knock-in, knock-down, and/or knock-out techniques. Production of the gene product can be influenced by the levels of translation factors available, by the presence of transcript-specific ribozymes, or using anti-sense technology. The activity of the gene product can be directly affected by addition of inhibitors or enhancers. Thus, the method used to manipulate the gene product can vary from assay to assay, depending upon the compound to be assayed and the gene product employed.

The assay solution is incubated with the sentinel molecule, and one or more test compounds to be screening for activity. The test compound can be a single compound, or a library of compounds to be screened for activity. Sources for test compounds include, but are not limited to, chemical catalogs such as those available from Sigma or Aldrich, and commercial libraries of compounds. Optionally, the test compound can be one or more antibiotic compounds. The addition of the test compound to the assay solution can increase the extent of modification or alter the type of modification that the sentinel molecule undergoes; alternatively, the compound being tested can inhibit or interfere with the modification of the compound.

Within the assay solution, the gene product is allowed to interact with the sentinel molecule. After incubation, the sentinel molecule is examined for modification by one or more analytical techniques. A change in the modification state of the sentinel molecule indicates that the compound or compounds being screened have activity with respect to the gene product and sentinel molecule used in the method.

Methods of Screening a Compound for Antibiotic Activity

Another embodiment of the methods of the present invention provides methods for screening a compound for antibiotic activity. The screening capitalizes on differences in cellular metabolism between targeted and non-targeted organisms, to identify compounds which detrimentally affect the targeted organism (generally, a pathogenic organism) without affecting or harming other, non-targeted cells (e.g., a host organism, or nonpathogenic cells existing in the same environment as the pathogenic cells). In one embodiment, the targeted organism includes prokaryotic cells, while the non-targeted organism are eukaryotic cells. In another embodiment, the targeted organism includes pathogenic eukaryotic cells (for example, yeast and fungi). Alternatively, this screening procedure can be used to identify antibiotics that, for example, affect the metabolism of certain classes of prokaryotes or microbes but not other classes. Thus, compounds with antibiotic activity can be identified that affect certain aspects of cellular metabolism, for example, the generation and recognition of modified tRNA molecules, which may differ among organisms.

These methods start with providing a cell line that has a sentinel molecule that is normally modified in the targeted cell, but not modified in the non-targeted cell (e.g., the host). The cell line can be modified, if necessary, to express one or more gene products needed to screen for the antibiotic compounds (for example, the cell line can be transformed with expression vectors that encode specific enzymatic activities).

During the methods of the present invention, the cell line is optionally lysed to produce a cellular extract. The sentinel molecule can be expressed in the cell line, or it can subsequently be added to the cells or cellular extract. Sentinel molecules which can be used in these methods include, but are not limited to, RNA molecules, DNA molecules, peptides, proteins, carbohydrates, lipids, naturally-occurring small molecule substrates, and synthetic small molecule substrates. Preferably, the sentinel molecule is an RNA molecule, such as a tRNA, rRNA, mRNA, guide RNA, snoRNA, snRNA, hnRNA, and the like. Alternatively, the sentinal molecule is a carbohydrate-based or lipid based molecule (e.g., phospholipids, triglycerides, lipopolysaccharides, glycolipids, glycoproteins, peptidoglycans, lipoproteins, mycolic acids, teichoic acids, teichuronic acids, lipoteichoic acids, and the like). The sentinel molecule can be a single type of molecule, or it can be a mixture of molecules. Using a mixture of sentinel molecules increases the number of gene products participating in the screening assay, and thus improves the likelihood of finding a test compound with activity.

The assay solution is incubated with the sentinel molecule, and one or more compounds to be screening for antibiotic activity. The compound to be screened can be a single compound, or a library of compounds. Sources for such test compounds include, but are not limited to, chemical catalogs such as those available from Sigma or Aldrich, and commercial libraries of compounds. The addition of the test compound to the assay solution can increase the extent of modification or alter the type of modification that the sentinel molecule undergoes; alternatively, the compound being tested can inhibit or interfere with the modification of the compound.

To screen the compound (or a mixture of compounds) for antibiotic activity, the compound is added to the cell line. After treating the cell line, the sentinel molecule is monitored for modification, thus determining whether the compound has an antibiotic activity. The monitoring for modification can be performed by any of the techniques as described previously, alone or in combination, or as otherwise known in the art.

In Vitro Methods for Identifying Gene Products involved in RNA Modification

Yet another embodiment of the methods of the present invention provides in vitro methods for identifying one or more gene products involved in RNA modification, as well as the genes that encode these gene products. The in vitro methods can include protocols in which the test gene activity is augmented, as well as protocols in which there is a reduction of the test gene activity. Both protocols are described below.

At least one cell having one or more test genes of interest is used in these in vitro methods. Current micro-manipulation technology and sensitive analytical techniques have made it possible to perform experiments on single cells. Alternatively, a group of cells or a cell line can be used. The cells can be prokaryotic cells or eukaryotic cells. The test gene or genes being examined for whether they encode one or more gene products involved in RNA modification can be part of the cellular genome, or they can be added to the cells, for example, in the form of expression vectors. In this manner, large numbers of nucleic acid segments, or libraries of genomic fragments, can be analyzed.

In addition, the expression level of the gene product(s) can be altered or manipulated. In the methods wherein augmentation of the test gene activity is desired, over-expression constructs can be used to produce elevated levels of the test gene activity and increased expression of the gene product(s). Alternatively, in protocols wherein the test gene activity is reduced, induction of a mechanism that reduces or eliminates the expression of the test gene is employed. If the test gene is involved in the modification of the sentinel RNA molecule, then the induced mechanism against the test gene will lead to a lessened state of modification on the sentinel RNA.

A cellular extract is prepared from the cell or cells, such that the extract contains the gene product(s) potentially having RNA modification activity. One or more sentinel RNA molecules are incubated with the cellular extract and gene products, during which time the sentinel RNA molecule can be modified by the gene product(s). Examples of sentinel RNA molecules which can be used in the present invention include, but are not limited to tRNA molecules, rRNA molecules, mRNA molecules, guide RNA molecules, snRNA molecules, snoRNA molecules, hnRNA molecules, and the like. Optionally, the sentinel RNA molecules are tRNA molecules. It should be noted that either a single type of sentinel RNA molecule, or a collection of sentinel RNA molecules, can be employed in the present invention.

After incubation, the sentinel RNA molecules are analyzed for one or more modifications. Determining whether the sentinel RNA molecule has been modified can be performed, e.g., by any of the techniques as described previously, alone or in combination, or as otherwise known in the art. The methods of the present invention can identify modified RNA molecules that have not previously been described. The in vitro method can further include the step of identifying the test gene or test genes that encode the gene products involved in RNA modification by methods, such as DNA sequencing, which are well known to one in the art. References describing nucleic acid sequencing techniques are known in the art, and include, for example, Berger and Kimmel, Innis, Sambrook and Ausubel, all supra.

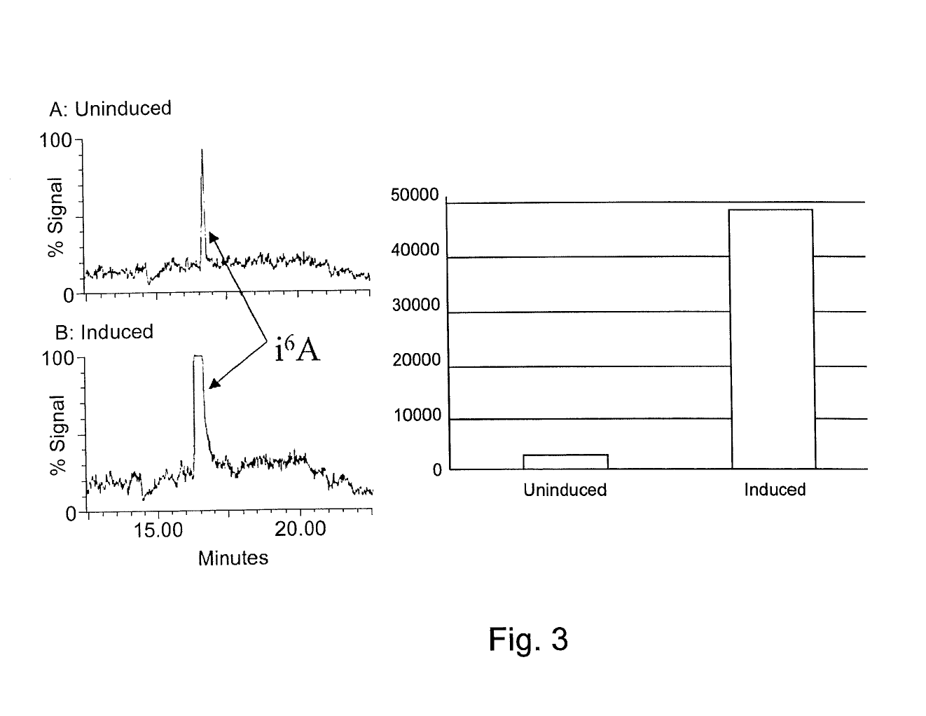

FIG. 3 depicts an in vitro assay as performed by the methods of the present invention. E. coli cells were transformed to over-express the miaA gene product (a tRNA isopentenyl pyrophosphate transferase; Leung et al. (1997) J Biol. Chem. 272:13073-83). The transferase catalyzes the addition of an isopentenyl group to certain adenosines (e.g., A37) adjacent to the anticodon region of some tRNA molecules, the first step in the biosynthesis of 2-methylthio-N6-(delta 2-isopentenyl)-adenosine. Lysate was prepared from the cells, and incubated with synthetic cysteine tRNA sentinal molecules and DMAPP (dimethylallyl diphosphate). After the substrate adenosines had been modified, the sentinal molecules were digested to component nucleosides and analyzed by LC/MS (using reverse-phase column chromatography on a C18 column (Higgins Analytical, Mountain View Calif.), and triple quadropole mass detection (Quattro II™, Micromass Inc, Beverly, Mass.) equipped with a Z Spray™ electrospray ionization source operated in positive ion mode (single ion recording mode), monitoring a mass to charge ratio range of about 100-450 daltons. Induction of transferase activity and concomitant increases in product i6A levels as measured by mass spectrometry data are shown in FIG. 3, panel A (“uninduced state”) and panel B (“induced state”). Relative peak areas of the i6A product are depicted in panel C. The data demonstrates the viability of this in vitro method for identifying one or more gene products involved in RNA modification (in this case, the known tRNA transferase).

In Vivo Methods for Identifying Gene Products involved in RNA Modification

A further embodiment of the methods of the present invention provides in vivo methods for identifying one or more gene products involved in RNA modification, as well as the genes that encode these gene products. The in vivo approach typically involves the induction of a mechanism that reduces or eliminates the expression of the test gene from the genome of the cell, leading to a reduction in the concentration of the gene product. If the test gene is involved in the modification of the sentinel RNA, then this chain of cellular events will lead to decreased modification of the sentinel RNA. Alternatively, the expression of the test gene can be induced, or increased, such that the sentinel RNA molecules are newly modified, or modified to a greater extent, as compared to in the uninduced state.

In the in vivo methods for identifying one or more gene products involved in RNA modification, a cell (or group of cells) is provided that expresses one or more sentinel RNA molecules, and has one or more test genes of interest. The cells can be bacterial cells, or they can be of eukaryotic origin. Sentinel RNA molecules that can be found within, or introduced into, the cell include, but are not limited to, tRNA molecules, rRNA molecules, mRNA molecules, guide RNA molecules, hnRNA molecules, snRNA molecules, snoRNA molecules, and the like. Optionally, the sentinel RNA molecules are tRNA molecules. It should be noted that either a single type of sentinel RNA molecule, or a group of sentinel RNA molecules, can be employed in the present invention.

The test gene or genes to be examined can be part of the cellular genome, or they can be added to the cellular environment in the form of expression vectors. Using such expression vectors, various nucleic acid segments of interest, or libraries of genomic fragments, can be analyzed. Alternatively, transcriptional elements and/or promoters can be inserted into the cell's genomic material, thus providing a means by which the expression of proximal nucleic acid sequences can be manipulated. Thus, the expression level of the gene product(s) encoded by either the introduced or transcriptionally-modified sequences can be altered or manipulated.

The one or more test genes of interest are manipulated within the cellular environment, such that expression of the test gene is altered. The mechanism used to alter the expression of the test gene can include, but is not limited to, the following techniques: targeted destruction of the RNA transcripts by gene-specific ribozymes; generation of temperature-sensitive mutations in the test gene that allow for temperature-dependent expression of the gene product; anti-sense technology; gene knock-out, knock-in, or knock-down technologies; or any other method known to those skilled in the art.

In the methods wherein augmentation of the test gene activity is desired, over-expression constructs can be used to produce elevated levels of the test gene activity and increased expression of the gene product(s). Alternatively, in protocols wherein the test gene activity is reduced, induction of a mechanism that reduces or eliminates the expression of the test gene is employed. If the test gene is involved in the modification of the sentinel RNA molecule, then the induced mechanism against the test gene will lead to a lessened state of modification on the sentinel RNA.

After the one or more test genes have been manipulated, and the resulting changes in gene product expression have occurred, the sentinel RNA molecules are analyzed for modification. The determining whether the sentinel RNA molecule has been modified can be performed by any of the techniques as described previously, alone or in combination, or as otherwise known in the art. The methods of the present invention can identify modified RNA molecules that have not previously been described. Furthermore, the in vivo method can also include the step of identifying the test gene or test genes that encode the gene products involved in RNA modification.

Identification of Genes Involved in RNA modification

The in vitro methods and the in vivo methods for identifying gene products can further include the step of identifying the gene that encodes the gene product. Techniques for identification of genes and/or nucleic acid sequences are known in the art. Sequencing and other standard recombinant techniques useful for the present invention can be found, for example, in Berger and Kimmel, Guide to Molecular Cloning Techniques, Methods in Enzymology volume 152 (Academic Press, Inc., San Diego, Calif.); Sambrook et al., Molecular Cloning—A Laboratory Manual (2nd Ed.), Vol. 1-3, (1989, Cold Spring Harbor Laboratory, Cold Spring Harbor, N.Y.) and Current Protocols in Molecular Biology, F. M. Ausubel et al., eds., Current Protocols, a joint venture between Greene Publishing Associates, Inc. and John Wiley & Sons, Inc., (supplemented through 1999).

Cloning and expression techniques which can be utilized in this step of the methods of the present invention have been described previously, with respect to expressing the gene product in the cell. These references also describe additional approaches which can be employed to determine the identity of the test genes of the present invention. In addition, in vitro amplification methods can also be used to amplify and/or sequence the test genes of interest. Examples of in vitro amplification and sequencing techniques, including the polymerase chain reaction (PCR) the ligase chain reaction (LCR), Qβ-replicase amplification and other RNA polymerase mediated techniques (e.g., NASBA) can be found in Berger, Sambrook, and Ausubel, id., as well as in Mullis et al., (1987) U.S. Pat. No. 4,683,202; PCR Protocols A Guide to Methods and Applications by Innis et al. eds. (1990, Academic Press Inc., San Diego, Calif.); Arnheim & Levinson (Oct. 1, 1990) C&EN 36-47; The Journal Of NIH Research (1991) 3, 81-94; Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564; Wallace et al., U.S. Pat. No. 5,426,039; Cheng et al. (1994) Nature 369: 684-685, and the references therein.

The function of a gene can be determined via over-expression of the encoded gene product in vivo using the methods of the present invention. A putative RNA modification gene (the test gene) is cloned into an expression vector plasmid having an inducible promoter (e.g., the E. coli lac promoter). Optionally, the plasmid also includes an antibiotic resistance gene (for example, beat-lactamase, which confers ampicillin resistance, or aminoglycoside 3′-phosphotransferase, which confers kanamycin resistance) to allow selection of plasmid-carrying cells and confirmation of stable transformation. In the plasmid, the test gene is operatively linked to the inducible promoter.

The expression vector comprising the gene is transferred into a bacterial cell line (e.g., E. coli) by methods familiar to those skilled in the art. References describing the techniques involved include Sambrook, supra and Ausubel, supra. As a control, the expression vector (sans the test gene) is transfected into additional cells and used to determine background levels of tRNA modifications.

The bacteria are allowed to grow in media containing the selected antibiotic and an agent that induces expression of the test gene (for example, the inducing agent IPTG can be employed for the lac operon promoter). The cells are cultured until the cell density indicates that the bacteria are in early log phase growth. Expression of the test gene is induced and the gene product is allowed to accumulate in the bacteria for a specified period of time, e.g., between 2 hours and overnight. The bacteria are then harvested (e.g., by centrifugation, filtration, or other means known in the art) and the cells are lysed. Lysis can be performed, e.g., with a solution containing chaotropic salts, such as guanidinium thiocyanate, phenol and detergent and buffered to pH of between 5.5 and 6.5 (see, for example, Berger and Kimmel, supra). The RNA is extracted from this mixture by the addition of chloroform and removal of the aqueous phase. The RNA is precipitated from the aqueous phase by the addition to this mixture of 2.5 volumes of neat ice cold ethanol followed by high speed centrifugation. The purified RNA is then dissolved in a buffered saline solution.

The tRNA can then be purified from the other cellular RNAs by, for example, gel filtration chromatography. See, for example, Reed et al. (1988) Cell 53:1949-1961. The tRNA-containing fractions of the eluate are pooled together and concentrated by extraction with neat 1-butanol. The tRNA is digested to nucleosides (or nucleotides) using nuclease P1, alkaline phosphatase and phosphonucleotide di-esterase (see, for example, Crain (1990) Methods in Enzymology 193:782-790 and Nishimura et al. (1997) Methods in Enzymology 155:373-379). The resulting nucleosides can be analyzed for a particular modification by one of several methods, depending on the nucleoside modification in question.

Using the methods of the present invention, resolution of many nucleosides is possible by HPLC alone using reversed phase chromatography on a C18 column. In the event that a particular modification of interest (i.e., a sentinel structure) is not resolved from other modifications, the use of LC-MS has been demonstrated to effectively detect modified nucleosides in mixed solutions (Pomerantz and McCloskey (1990) Methods in Enzymology 193:796-824). A measured increase in the amount of a modified nucleoside indicates that the gene is involved in the pathway of tRNA modification.

High Throughput Methodology

Optionally, one or more detection techniques that will allow for the preparation and rapid analysis of the sentinel molecules can be employed in the methods of the present invention. Techniques for the growth of bacteria in multi-well plates and transformation of cells within multi-well plates have been described elsewhere These techniques can be employed in the methods of the present invention.

Sentinel molecules such as tRNA from bacterial cells and cell lysates can be prepared in a parallel fashion for mass spectroscopy, LC/MS, LC-NMR or other analytical instrumentation in a parallel fashion using multi-well plates. Multi-well plates having 96, 384, 768 or 1536 wells are available from various suppliers such as VWR Scientific Products (West Chester, Pa.). Instrumentation for autosampling from 96-well plates (or other formats) can be used to transfer samples from the multi-well plates to, for example, the mass spectrometer; this instrumentation is available from several sources. Thus, by using a multi-well format, the methods of the present invention can be performed in a parallel high-throughput format.

The described procedure allows for the discovery of the gene encoding the enzymatic activity responsible for lysidinylation of cytosine as found in the isoleucyl tRNA of bacteria.

The methodologies for structural analysis can be used to discover new covalent modifications in bacteria of both eubacterial and archeae origins and to further identify the genes in these organisms that encode the enzymes that produce the covalent modifications.

Data Analysis

The step of detecting the presence or absence of one or modifications to the sentinal molecule can further include analyzing data generated during the detection process. For example, data generated during mass spectrometry analysis can be quantitated and compared between samples containing the test gene product and control samples, by methods known to one in the art. Additional examples of data analysis are provided in, for example, provisional application 60/225,506 (filed Aug. 15, 2000) and copending application (Attorney Docket No. 16-000220US, filed Feb. 23, 2001). Furthermore, the methods of the present invention can be automated, for example, for generation and analysis of data collected using high throughput methodologies. Instruction sets for analyzing the results generated by the methods of the present invention can be constructed by one of skill using a standard programming language such as C, C++, Visual Basic, Fortran, Basic, Java, or the like. For example, a system for use with the methods of the present invention can include one or more of the following: a device for providing and/or sorting the libraries of nucleic acids used in the methods; a device for incubating the assay compositions with the sentinal molecules; a device for analyzing the signals from the modified sentinal molecules; a computer or computer-readable medium; software for analyzing the presence or absence of one or more modifications; software for picking “hits” from any expression library (e.g., library members which encode enzymes that are relevant to sentinal molecule modification) and, optionally, for sequencing or otherwise analyzing the hits; and a user interface (e.g., a GUI in a standard operating system such as a Windows, Macintosh, UNIX, LINUX, and the like). Standard desktop applications which can be employed with one or more of these devices includes, but is not limited to, word processing software (e.g., Microsoft Word™ or Corel WordPerfect™), spreadsheet and/or database software (e.g., Microsoft Excel™, Corel Quattro Pro™, Microsoft Access™, Paradox™, Filemaker Pro™, Oracle™, Sybase™, and Informix™) and the like, which can be adapted for these (and other) purposes. Optionally, the computer or computer readable medium can provide the examination results in the form of an output file.

Uses of the Methods, Devices and Compositions of the Present Invention

Modifications can be made to the method and materials as described above without departing from the spirit or scope of the invention as claimed, and the invention can be put to a number of different uses, including:

The use of any method herein, to identify a gene encoding an RNA modification enzyme.

The use of a method or an integrated system to identify a gene encoding an RNA modification enzyme.

An assay, kit or system utilizing a use of any one of the selection strategies, materials, components, methods or substrates hereinbefore described. Kits will optionally additionally comprise instructions for performing the methods or assays, packaging materials, one or more containers which contain assay, device or system components, or the like.

In an additional aspect, the present invention provides kits embodying the methods and devices herein. Kits of the invention optionally comprise one or more of the following: (1) a library of nucleic acid sequences, optionally incorporated into expression vectors; (2) one or more sentinal molecules, such as RNA sentinal molecules; (3) one or more assay components, including, but not limited to buffers, substrates, cofactors, inhibitors, selection agents, antibiotics, enzymes, and the like; (4) a computer or computer-readable medium for performing the methods of the present invention and/or for storing the assay results; (5) instructions for practicing the methods described herein; and, optionally, (6) packaging materials.

In a further aspect, the present invention provides for the use of any component or kit herein, for the practice of any method or assay herein, and/or for the use of any apparatus or kit to practice any assay or method herein.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of identifying a gene encoding an RNA modification enzyme (RME), the method comprising:
providing a library of nucleic acids encoding one or more putative RMEs and expressing the library of nucleic acids to provide a plurality of putative RME compositions, wherein each of the putative RME compositions comprises one or more putative RME expression products;
incubating the plurality of putative RME compositions with one or more sentinel RNA molecules; and,
detecting the presence or absence of one or more modification to one or more of the sentinel RNA molecules, thereby determining that the putative RME has one or more RNA modification activities.

2. The method of claim 1, wherein providing a library of nucleic acids comprises providing a library of bacterial genes.

3. The method of claim 1, wherein providing a library of nucleic acids comprises providing synthesized nucleic acids.

4. The method of claim 1, wherein providing a library of nucleic acids comprises providing a plurality of expression vectors comprising the library of nucleic acids and transfecting one or more cells with the plurality of expression vectors.

5. The method of claim 1, wherein expressing the library of nucleic acids comprises inducing or increasing the expression of the one or more test genes.

6. The method of claim 1, wherein expressing the library of nucleic acids comprises reducing or eliminating the expression of the one or more test genes.

7. The method of claim 1, wherein the one or more sentinel RNA molecules comprises one or more of a tRNA molecule, an rRNA molecule, a mRNA molecule, a guide RNA molecule, an hnRNA molecule, a snRNA molecule, a snoRNA molecule, or a combination thereof.

8. The method of claim 1, wherein incubating the plurality of putative RME compositions further comprises adding one or more substrates, cofactors or inhibitors to the plurality of putative RME compositions.

9. The method of claim 8, wherein the one or more substrates comprise nucleosides.

10. The method of claim 8, wherein the one or more substrates comprise one or more purified small molecule substrates.

11. The method of claim 8, wherein the one or more substrates comprise one or more synthetic RNA substrates.

12. The method of claim 8, wherein the one or more cofactors comprise one or more feedstock lysates.

13. The method of claim 1, wherein detecting the presence or absence of one or more modification comprises performing mass spectrometry, thin layer chromatography, size-exclusion chromatography, HPLC, capillary electrophoresis, NMR spectroscopy, or a combination thereof.

14. The method of claim 1, wherein detecting the presence or absence of one or more modification comprises performing LC/MS.

15. A method of testing a putative RME for activity on a sentinel RNA molecule, the method comprising:
selecting a putative RME or a nucleic acid which encodes the putative RME;
incubating a composition comprising the RME and a sentinel RNA; and,
detecting the presence or absence of one or more modifications to the sentinel RNA, thereby determining that the putative RME has one or more RNA modification activities.

16. The method of claim 15, wherein selecting a putative RME further comprises expressing the nucleic acid which encodes the putative RME.

17. The method of claim 16, wherein expressing the nucleic acid comprises inducing or increasing the expression of the nucleic acid.

18. The method of claim 16, wherein expressing the nucleic acid comprises reducing or eliminating the expression of the nucleic acid.

19. The method of claim 15, wherein the composition comprising the RME and a sentinel RNA further comprises one or more substrates, cofactors or inhibitors.

20. The method of claim 15, wherein detecting the presence or absence of one or more modifications comprises performing mass spectrometry, thin layer chromatography, size-exclusion chromatography, HPLC, capillary electrophoresis, NMR spectroscopy, or a combination thereof.

21. The method of claim 15, wherein detecting comprises performing LC/MS.

22. The method of claim 15, further comprising identifying the nucleic acid which encodes the putative RME.

23. The nucleic acid which encodes the putative RME, as identified by the method of claim 22.

24. A method of screening a potential RME modulator composition for one or more RME modulation activities, comprising
providing an RME and a sentinel RNA molecule;
incubating the RME and the sentinel RNA molecule with a potential RME modulator composition;
determining whether the sentinel RNA molecule was modified by the RME in the presence of the potential RME modulator composition to produce a modified sentinel RNA molecule, thereby screening the potential RME modulator composition for one or more RME modulation activities.

25. The method of claim 24, wherein providing an RME comprises providing a vector comprising a gene encoding the RME, expressing the vector in a biological system to produce the RME, and preparing an assay solution from the biological system and containing the RME.

26. The method of claim 25, wherein the biological system comprises a cell.

27. The method of claim 25, wherein the vector further comprises a gene encoding the sentinel RNA molecule.

28. The method of claim 25, wherein expressing the vector in a biological system comprises increasing or reducing the expression level of the gene encoding the RME.

29. The method of claim 24, wherein the RME comprises an enzyme or a catalytic RNA.

30. The method of claim 24, wherein the potential RME modulator composition comprises one or more antibiotic compounds.

31. The method of claim 24, wherein determining whether the sentinel molecule was modified comprises performing mass spectrometry, thin layer chromatography, size-exclusion chromatography, HPLC, capillary electrophoresis, NMR spectroscopy, or a combination thereof.

32. The method of claim 24, wherein the sentinel RNA molecule comprises one or more of a tRNA molecule, an rRNA molecule, a mRNA molecule, a guide RNA molecule, an hnRNA molecule, a snRNA molecule, a snoRNA molecule, or a combination thereof..

33. The method of claim 24, wherein the sentinel molecule comprises one or more tRNA molecules.

34. The method of claim 24, wherein the modified sentinel RNA molecule comprises one or more of a methylated, alkylated, acetylated, esterified, ubiquitinated, lysinylated, phosphorylated, sulfated, or glycosylated sentinel RNA molecule.

35. A RME modulator composition, as screened by the method of claim 24.

36. A modified sentinel RNA molecule, as determined by the method of claim 24.

37. An in vitro method for identifying one or more gene products involved in RNA modification, the method comprising:
providing at least one cell comprising one or more test genes that encode a gene product;
preparing a cellular extract from the at least one cell, wherein the cellular extract comprises the gene product;
incubating the cellular extract with a sentinel RNA molecule; and
determining whether the sentinel RNA molecule has been modified by the gene product, thereby identifying the gene product involved in modifying the sentinel RNA molecule.

38. The in vitro method of claim 37, wherein providing at least one cell further comprises altering the expression of the test gene.

39. The in vitro method of claim 38, wherein altering the expression of the test gene comprises inducing or increasing the expression of the test gene.

40. The in vitro method of claim 38, wherein altering the expression of the test gene comprises reducing or eliminating the expression of the test gene.

41. The in vitro method of claim 37, wherein preparing the cellular extract comprises preparing cellular extracts from at least one uninduced cell and preparing cellular extracts from at least one induced cell.

42. The in vitro method of claim 37, wherein providing at least one cell comprises providing one or more cell lines with particular genotypes.

43. The in vitro method of claim 37, wherein providing at least one cell comprises manipulating one or more cell lines to produce one or more particular phenotypes.

44. The in vitro method of claim 37, wherein the at least one cell comprises a bacterial cell.

45. The in vitro method of claim 37, wherein the at least one cell comprises a eukaryotic cell.

46. The in vitro method of claim 37, wherein the sentinel RNA molecule comprises one or more of a tRNA molecule, an rRNA molecule, a mRNA molecule, a guide RNA molecule, an hnRNA molecule, a snRNA molecule, a snoRNA molecule, or a combination thereof.

47. The in vitro method of claim 37, wherein determining whether the sentinel RNA molecule has been modified comprises performing mass spectrometry, thin layer chromatography, HPLC, capillary electrophoresis, NMR spectroscopy, X-ray crystallography, cryo-electron microscopic analysis, or a combination thereof.

48. The in vitro method of claim 37, further comprising identifying the at least one test gene that encodes the gene product.

49. A gene product involved in RNA modification, as identified by the in vitro method of claim 37

50. A gene encoding the gene product of claim 49.

51. A sentinel RNA molecule, as modified by the gene product of claim 49.

52. An in vivo method for identifying one or more gene products involved in RNA modification, the method comprising:
providing a cell comprising at least one sentinel RNA molecule, and one or more test genes that encode one or more gene products;
manipulating the one or more test genes within the cell; and
monitoring the sentinel RNA molecule for modification by the one or more gene products, thereby identifying whether the one or more gene products are involved in RNA modification.

53. The in vivo method of claim 52, wherein providing the at least one cell comprises providing a bacterial cell.

54. The in vivo method of claim 52, wherein manipulating the one or more test genes comprises inducing or increasing the expression of at least one of the one or more test genes.

55. The in vivo method of claim 52, wherein manipulating the one or more test genes comprises reducing or eliminating the expression of at least one of the one or more test genes.

56. The in vivo method of claim 52, further comprising detecting an expression level of the one or more gene products.

57. The in vivo method of claim 56, wherein detecting the expression level of the one or more gene products comprises determining whether the expression level is increased, reduced or eliminated.

58. The in vivo method of claim 52, wherein monitoring the sentinel RNA molecule comprises performing mass spectrometry, thin layer chromatography, HPLC, capillary electrophoresis, NMR spectroscopy, X-ray crystallography, cryo-electron microscopic analysis, or a combination thereof.

59. The one or more gene products involved in RNA modification, as identified by the method of claim 52.

60. The one or more test genes encoding the one or more gene products of claim 59.

61. The sentinel RNA molecule, as modified by the in vivo method of claim 52.

62. A method for screening a test compound for activity, the method comprising:
preparing an assay solution comprising a gene product which modifies a sentinel molecule;
incubating the assay solution with the sentinel molecule and the test compound; and
determining whether the sentinel molecule was modified by the gene product in the presence of the test compound to produce a modified sentinel molecule, thereby screening the test compound for activity.

63. The method of claim 62, wherein preparing the assay solution comprises preparing a cellular extract.

64. The method of claim 62, wherein preparing the assay solution comprises:
providing at least one cell comprising one or more genes that encode the gene product which modifies a sentinel molecule;
altering an expression level of the one or more genes that encode the gene product; and
lysing the at least one cell, thereby preparing the assay solution.

65. The method of claim 64, wherein providing at least one cell comprises providing at least one bacterial cell or at least one eukaryotic cell.

66. The method of claim 64, wherein altering the expression level comprises increasing or reducing the expression level.

67. The method of claim 62, wherein the gene product comprises an enzyme or a catalytic RNA.

68. The method of claim 62, wherein the test compound comprises one or more antibiotic compounds.

69. The method of claim 62, wherein determining whether the sentinel molecule was modified comprises performing mass spectrometry, thin layer chromatography, HPLC, capillary electrophoresis, NMR spectroscopy, X-ray crystallography, cryo-electron microscopic analysis, or a combination thereof.

70. The method of claim 62, wherein the sentinel molecule comprises one or more of: RNA molecules, DNA molecules, peptides, proteins, carbohydrates, lipids, naturally-occurring small molecule substrates, and synthetic small molecule substrates.

71. The method of claim 70, wherein the RNA molecules comprise one or more of tRNA molecules, rRNA molecules, mRNA molecules, guide RNA molecules, snRNA molecules, snoRNA molecules, or hnRNA molecules.

72. The method of claim 70, wherein the sentinel molecule comprises one or more tRNA molecules.

73. The method of claim 62, wherein the modified sentinel molecule comprises one or more of a methylated, alkylated, acetylated, esterified, ubiquitinated, lysinylated, phosphorylated, sulfated, or glycosylated sentinel molecule.

74. A test compound, as screened by the method of claim 62.

75. A modified sentinel molecule, as determined by the method of claim 62.

76. A method for screening a compound for antibiotic activity, the method comprising:
providing a cell line comprising a sentinel RNA molecule that is normally modified in a prokaryotic system, but not modified in a eukaryotic system;
treating the cell line with the compound; and
monitoring the sentinel RNA molecule for modification, thereby screening the compound for antibiotic activity.

77. The method of claim 76, wherein providing the cell line comprises providing a bacterial cell line or a eukaryotic cell line.

78. The method of claim 76, wherein providing the cell line comprises providing a cellular extract.

79. The method of claim 76, wherein the sentinel RNA molecule comprises a tRNA molecule, an rRNA molecule, a mRNA molecule, a guide RNA molecule, a snRNA molecule, a snoRNA molecule, an hnRNA molecule, or a combination thereof.

80. The method of claim 76, wherein monitoring the sentinel RNA molecule for modification comprises performing mass spectrometry, thin layer chromatography, HPLC, capillary electrophoresis, NMR spectroscopy, X-ray crystallography, cryo-electron microscopic analysis, or a combination thereof.

81. The method of claim 80, wherein the RNA modification comprises one or more of methylation, alkylation, acetylation, esterification, ubiquitination, lysinylation, phosphorylation, sulfation, or glycosylation of the sentinel RNA molecule.

82. An antibiotic as identified by the method of claim 76.

83. A modified sentinel molecule, as determined by the method of claim 76.

84. A method of identifying a gene encoding a desired gene product, the method comprising:
providing a library of nucleic acids and expressing the library of nucleic acids to provide a plurality of gene products;
incubating the plurality of gene products with one or more sentinel molecules; and,
detecting the presence or absence of one or more modification to one or more of the sentinel molecules, thereby determining that the plurality of gene products comprises one or more desired gene products and identifying the gene encoding the desired gene product.
